(19) United States
(12) Patent Application Publication
(10) Pub. No.: US 2012/0208924 A1
(43) Pub. Date: Aug. 16, 2012

(54) EPOXY RESIN COMPOSITION

(75) Inventors: Bandeep Singh, Wytheville, VA (US); Cherif Ghoul, Mulhouse (FR); Stéphane Schaal, Sierentz (FR); Prateek Puri, Dwarka (IN)

(73) Assignee: ABB RESEARCH LTD, Zurich (CH)

(21) Appl. No.: 13/383,774

(22) PCT Filed: Jul. 14, 2009
(86) PCT No.: PCT/EP2009/058965

§ 371 Date: Apr. 26, 2012

(51) Int. Cl. H01B 3/40; C07C 69/753; C08L 63/00

(52) U.S. Cl. 523/400; 560/127

(57) ABSTRACT

An exemplary curable epoxy resin composition is disclosed which includes at least an epoxy resin component and a hardener component, and optionally further additives, wherein (a) at least a part of the hardener component is a chemically modified polycarbonic acid anhydride; (b) an optional glycol or polyglycol; (c) or a compound containing two carboxylic groups is included; and (d) the chemically modified acid anhydride hardener is present in an amount comprising at least 10% of reactive hardening groups calculated to all the reactive hardening groups contained in the total amount of hardener component present in the epoxy resin composition.

### FIELD OF THE INVENTION

The present invention refers to a curable epoxy resin composition comprising an epoxy resin component and a hardener component, wherein at least a part of the hardener component is a chemically modified polycarbonic acid anhydride, said chemically modified polycarbonic acid anhydride being the reaction product of a polycarbonic acid anhydride and a glycol or a poly-glycol, or a reaction product of a polycarbonic acid anhydride and a compound containing two carboxylic groups. The resulting cured epoxy resin composition has an improved low temperature cracking resistance to temperatures down to about minus 70° C. (−70° C.) and is suitable for the use as an encapsulating material for electrical applications, especially for applications where the material is subjected to thermal shocks such as metal core-coil assemblies of instrument transformers.

### STATE OF THE ART

Cracking resistance of electrical encapsulating materials based on cured epoxy resin compositions, especially at low temperatures, is an important technical problem. Encapsulating insulation materials in electrical applications surrounding metal core-coil assemblies in electrical instrument transformers, such as insulations made from epoxy resin compositions, are prone to cracking at low temperatures. This is mainly due to the difference in the Coefficient of Thermal Expansion (CTE) of the epoxy insulation system which generally is comparatively high and the Coefficient of Thermal Expansion (CTE) of the metal core-coil assembly which is comparatively low. Conventional epoxy resin compositions being used as encapsulants for electrical equipment usually only insufficiently fulfill the low temperature cracking requirements. Different solutions have been suggested to solve this problem.

U.S. Pat. No. 3,926,904 and U.S. Pat. No. 5,939,472 suggest rubber inclusions in the epoxy resin composition to improve the thermal shock resistance of the insulator. U.S. Pat. No. 4,285,853 and U.S. Pat. No. 5,985,956 disclose the use of nanoclay such as Montmorillonite and Wollastonite along with a silica filler in epoxy resin compositions. Nanoclays lower the overall Coefficient of Thermal Expansion (CTE) of the cured epoxy resin composition which improves their low temperature cracking resistance. However, the major shortcoming of this technique is the difficulty in exfoliating the nanoclay particles for obtaining a sufficiently increased surface area contact and maximum CTE reduction. In addition, this method of nanoclay inclusion is not a cost-effective measure for enhancing low temperature cracking resistance. Inclusion of further components into the epoxy resin composition is technically difficult and generally changes the physical properties of the epoxy resin composition and is cost intensive.

### SUMMARY OF INVENTION

It has now been found that curable epoxy resin compositions with improved low temperature cracking resistance down to temperatures of about minus 70° C. (−70° C.) can be obtained without additional inclusion of a compound not generally used in curable epoxy resin compositions. This improved low temperature cracking resistance is a special advantage compared to the conventional anhydride cured epoxy resin compositions, based e.g. on diglyci-dylether-bisphenol compounds such as diglycidylether-bisphenol A (DGEBA), and a phthalic acid anhydride hardener such as methyl-tetrahydrophthalic anhydride (MTHPA). In addition, according to the present invention these cracking resistance properties are obtained at low cost, with no loss to the APG (Automated Pressure Gelation) processability or to the vacuum casting process of the curable epoxy resin composition, whereby the cured epoxy resin composition retains similar mechanical, thermal ageing and dielectric properties as measured for conventional epoxy resin compositions.

This is achieved by using a curable epoxy resin composition comprising at least one epoxy resin component together with at least one hardener component, wherein at least a part of the hardener component is a chemically modified polycarbonic acid anhydride, said chemically modified polycarbonic acid anhydride being the reaction product of a polycarbonic acid anhydride and a glycol (diol) or a polyglycol, or being the reaction product of a polycarbonic acid anhydride and a compound containing two carboxylic groups. Said hardener component is prepared separately and then added to the epoxy resin composition. The epoxy resin composition may contain further known additives. Said composition satisfies the cost constraints and is suitable for the use as an encapsulating material for electrical applications, especially for applications where the material is subjected to thermal shocks at low temperatures such as metal coils, cores and auxiliary parts of instrument transformers.

The present invention is defined in the claims. The present invention refers to a curable epoxy resin composition comprising at least an epoxy resin component and a hardener component, and optionally further additives, characterized in that
(a) at least a part of the hardener component is a chemically modified polycarbonic acid anhydride, said chemically modified polycarbonic acid anhydride being the reaction product of a polycarbonic acid anhydride and a glycol (diol) or a polyglycol, or the reaction product of a polycarbonic acid anhydride and a compound containing two carboxylic groups;
(b) said glycol or polyglycol being selected from the group comprising compounds of formula (I):

HO—(CₙH₂ₙ—O)ₘ—H (I)
wherein
n is an integer from 2 to 8; m is an integer from 1 to 10;
(c) said compound containing two carboxylic groups being selected from the group comprising compounds of formula (II):

HOOC—(CₚH₂ₚ)—COOH (II)
wherein p an integer from 1 to 18; and
(d) the chemically modified acid anhydride hardener is present in an amount comprising at least 10% of the reactive hardening groups calculated to all the reactive hardening groups contained in the total amount of hardener component present in the epoxy resin composition.

The present invention further refers to a method of producing said curable epoxy resin composition. The present invention further refers to the use of said curable epoxy resin composition for the production of insulation systems in electrical articles.

The present invention further refers to the cured epoxy resin composition, which is present in the form of an electrical insulation system, resp. in the form of an electrical insulator. The present invention further refers to the electrical articles comprising an electrical insulation system made according to the present invention.

The present invention further refers to the chemically modified polycarbonic acid anhydride hardener compound and to a method of producing said chemically modified polycarbonic acid anhydride hardener compound. Said chemically modified polycarbonic acid anhydride hardener is prepared separately and prior to being added as a component to the epoxy resin composition.

The epoxy resin component present in the curable epoxy resin composition contains at least two 1,2-epoxy groups per molecule. Cycloaliphatic and aromatic epoxy resin compounds useful for the present invention comprise unsubstituted glycidyl groups and/or glycidyl groups substituted with methyl groups. These glycidyl compounds have an epoxy value (equiv./kg) preferably of at least three, preferably at least four and especially at about five or higher, preferably about 5.0 to 6.1. Preferred are for example optionally substituted epoxy resins of formula (III):

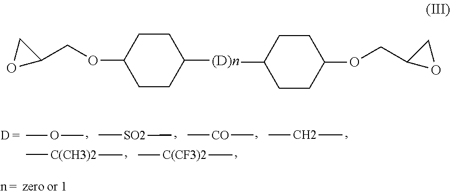

or optionally substituted epoxy resins of formula (IV):

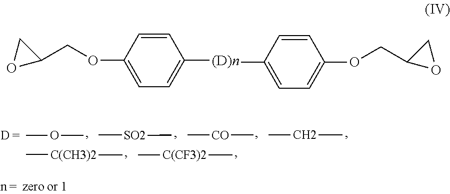

Compounds of formula (III) or formula (IV) wherein D is —(CH₂)— or [—C(CH₃)₂—] are preferred. Preferred further are aromatic compounds of formula (IV) wherein D is [—(CH₂)—] or [—C(CH₃)₂—], and preferably [—C(CH₃)₂—], i.e. diglycidylether of 2,2-bis-(4-hydroxyphenyl)-propane [diglycidylether of bisphenol A (DGEBA)]. DGEBA is commercially available as an epoxy resin component, e.g. as Epilox A19-00 (Leuna Harze GmbH.) or similar products. DGEBA as preferably used in the present invention has an epoxy value (equiv./kg) of at least three, preferably at least four and especially at about five or higher, preferably about 5.0 to 6.1.

Preferred cycloaliphatic epoxy resin compounds are for example Araldite® CY 184 (Huntsman Advanced Materials Ltd.), a cycloali-phatic diglycidylester epoxy resin compound having an epoxy content of 5.80-6.10 (equiv/kg) or Araldite® CY 5622 (Huntsman Advanced Materials Ltd.), a modified diglycidylester epoxy resin compound having an epoxy content of 5.80-6.10 (equiv/kg). Araldite® CY 5622 is a hydrophobic cycloaliphatic epoxy formulation for hydrophobicity transfer and recovery in outdoor epoxy resin compositions. A hydrophobic cycloaliphatic epoxy formulation means that the filler material has been pre-treated with a silane or a silane additive has been added to the composition.

Further epoxy resins to be used within the scope of the present invention are for example hexahydro-o-phthalic acid-bis-glycidyl ester, hexahydro-m-phthalic acid-bis-glycidyl ester or hexa-hydro-p-phthalic acid-bis-glycidyl ester.

Preferred epoxy resin compounds are liquid at room temperature or when heated to a temperature of up to about 65° C.

At least a part of the hardener component is a chemically modified polycarbonic acid anhydride which has been prepared prior to adding to the epoxy resin composition. Such chemically modified anhydride hardeners are preferably made from aliphatic and cycloaliphatic polycarbonic acid anhydrides, preferably from phthalic anhydride, methylhydrophthalic anhydride, and methyl-tetrahydrophthalic anhydride (MTHPA), by reaction with a compound of formula (I) or formula (II).

In said glycol or polyglycol of formula (I), n preferably is 2, 3, 4, 5 or 6, preferably 2, 3 or 5, preferably 3 or 5, preferably 5; and m preferably is 1 to 8; preferably 1 to 6, preferably 1 to 4, preferably 1 or 2, preferably 1.

In said compound of formula (II) containing two carboxylic groups, p preferably 2 to 16, preferably 2 to 10, preferably 2 to 8, preferably 2, 4, 5, 7, 8, preferably 2, 4, 7 or 8; and preferably 2, 4 or 7.

The chemically modified hardener component, therefore, is a reaction product obtained by reacting an aliphatic and cycloaliphatic polycarbonic acid anhydride with a glycol (diol) or polyglycol of formula (I), or with a compound containing two carboxylic groups of formula (II), prior to mixing with the epoxy resin component. The reaction is carried out by mixing the aliphatic and cycloaliphatic polycarbonic acid anhydride with a glycol (diol) or polyglycol of formula (I), or with a compound containing two carboxylic groups of formula (II), and heating the mixture to a temperature within the range of about 60° C. to about 100° C., preferably within the temperature range of about 75° C. to about 90° C., and preferably to about 85° C., for a time long enough until the diester with two active sites has formed. This generally is the case after heating for about 30 minutes to two hours.

The chemically modified hardener component is obtained by reacting a polycarbonic anhydride compound with a glycol of formula (I), in a molar ratio of preferably at least 2:1. The reaction can be illustrated as shown in Scheme 1, whereby the compound of formula (VI) is obtained.

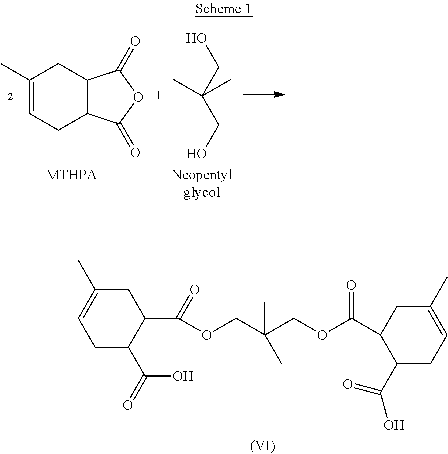

As can be seen, the reaction product contains two residues of the anhydride compound being bound to and bridged by the neopentyl glycol residue. This is the preferred reaction product. Analogously when the anhydride compound is reacted with a compound of formula (II), the reaction product, representing the modified hardener component, contains two residues of the anhydride compound being bound to and bridged by the residue of the compound of formula (II).

In this sense the modified hardener component according to the present invention preferably corresponds to the formula (VII) or to formula (VIII):

anhydride residue-O—(CₙH₂ₙ—)ₘ-anhydride residue (VII)

or

anhydride residue-O(O)C—(CₚH₂ₚ)—C(O)O-anhydride residue (VIII)

wherein n, m and p have the same meaning as given above.

The total amount of hardener component is composed of a chemically non-modified hardener compound and the chemically modified aliphatic and/or cycloaliphatic polycarbonic acid anhydride as defined above. The chemically non-modified hardener compound preferably is an aliphatic and/or cycloaliphatic polycarbonic acid anhydride, preferably phthalic anhydride, methylhydrophthalic anhydride and/or methyltetrahydrophthalic anhydride (MTHPA) as defined above.

The total amount of hardener component containing the reactive hardening groups is present within the epoxy resin composition in a concentration within the range of 0.2 to 1.2 equivalents of reactive hardening groups per epoxy equivalent present in the epoxy resin composition, resp. in the epoxy component(s), preferably in a concentration within the range of 0.8 to 1.2 equivalents of reactive hardening groups per epoxy equivalent present in the epoxy resin composition, and preferably in a concentration of about one equivalent of the reactive hardening group per one epoxy equivalent of the epoxy component. The expression “reactive hardening group” means a carboxy-anhydride group as contained e.g. in phthalic anhydride, or a reactive carboxyl group as contained in the modified hardener compound.

The chemically modified acid anhydride hardener is present preferably in an amount comprising 10% to 100%, preferably 20% to 90%, preferably 20% to 70%, preferably 30% to 70%, and most preferably 50% to 60% of the reactive hardening groups calculated to all the reactive hardening groups contained in the total amount of hardener component present in the epoxy resin composition.

The preferred method of making the hardener component being composed of the chemically non-modified hardener compound and the chemically modified aliphatic and/or cycloaliphatic polycarbonic acid anhydride, is characterized by the step of reacting at least 50%, preferably at least 70%, preferably at least 80% and preferably 100% of the chemically non-modified hardener compound, said chemically non-modified hardener compound being an aliphatic and/or cycloaliphatic polycarbonic acid anhydride as defined above, with a compound of formula (I) or with a compound of formula (II), wherein the hydroxyl-equivalents of the compound of the formula (I) or the carboxyl-equivalents of the compound of the formula (II) present within the reaction mixture are within the range of 10% to 100%, preferably within the range of 20% to 90%, preferably within the range of 20% to 70%, preferably within the range of 30% to 70%, and most preferably within the range of 50% to 60%, calculated to the total amount of reactive hardening groups present within the total amount of hardener component.

Methyltetrahydrophthalic anhydride (MTHPA) is commercially available and exists in different forms, e.g. as 4-methyl-1,2,3,6-tetrahydrophthalic anhydride or as 4-methyl-3,4,5,6-tetrahydrophthalic anhydride. Although the different forms are not critical for the application in the present invention, 4-methyl-1,2,3,6-tetrahydrophthalic anhydride and 4-methyl-3,4,5,6-tetrahydrophthalic anhydride are the preferred compounds to be used.

Methyltetrahydrophthalic anhydride (MTHPA) is often supplied commercially as a mixture containing MTHPA isomers as the main component, together with other anhydrides, such as tetrahydrophthalic anhydride (THPA), methylhexahydrophthalic anhydride (MHHPA) and/or phthalic anhydride (PA). Such mixtures may also be used within the scope of the present invention. The content of MTHPA within such a mixture is preferably at least 50% by weight, preferably at least 60% by weight, preferably at least 70% by weight, preferably at least 80% by weight, and preferably at least 90% by weight, calculated to the total weight of the anhydride mixture.

Examples of glycols and polyglycols falling under the scope of formula (I) are ethylene glycol, propylene glycol, butylenes glycol, pentylene glycol, neopentyl glycol, and the corresponding polyglycols with preferred molecular weights within the range of 200 to 2000, preferably from 200 to 1000, such as polyethylene glycol, polypropylene glycol, polybutylenes glycol, polypentylene glycol, polyneopentyl glycol. Preferred are polypropylene glycol (PPG) and neopentyl glycol (NPG). Most preferred is neopentyl glycol.

The compound containing two carboxylic groups as defined in formula (II) is preferably a compound of formula (IIa):

HOOC—(CH₂)ₚ—COOH (IIa)

wherein p has the meaning as given above. Examples of compounds containing two carboxylic groups falling under the scope of formula (II) are succinic acid (p=2), adipic acid (p=4), azelaic acid (p=7), sebacinic acid (p=8). A preferred diacid is azelaic acid (1,7-heptane dicarboxylic acid).

The curable epoxy resin composition comprising an epoxy resin component and a hardener component as defined above, may comprise further a filler material, preferably a mineral filler, and a curing agent for enhancing the polymerization of the epoxy resin with the hardener and further one or more optional additives selected from hydrophobic compounds including silicones, wetting/dispersing agents, plasticizers, antioxidants, light absorbers, pigments, flame retardants, fibers and other additives generally used in electrical applications. These are known to the expert.

The mineral filler is preferably selected from conventional filler materials as are generally used as fillers in electrical insulations. Preferably said filler is selected from the group of filler materials comprising inorganic oxides, inorganic hydroxides and inorganic oxyhydroxides, preferably silica, quartz, known silicates, aluminium oxide, aluminium trihydrate [ATH], titanium oxide or dolomite [CaMg(CO₃)₂], metal nitrides, such as silicon nitride, boron nitride and aluminium nitride or metal carbides, such as silicon carbide. Preferred are silica and quartz, specifically silica flour, with a SiO₂-content of about 95-98% by weight.

The mineral filler has an average grain size as known for the use in electrical insulation systems and is generally within the range of 10 micron up to 3 mm. Preferred, however, is an average grain size (at least 50% of the grains) within the range of about 1 μm to 300 μm, preferably from 5 μm to 100 μm, or a selected mixture of such average grain sizes. Preferred also is a filler material with a high surface area.

The inorganic filler is present in the epoxy resin composition, depending on the final application of the epoxy resin composition, preferably within the range of about 50% by weight to about 80% by weight, preferably within the range of about 60% by weight to about 75% by weight, and preferably at about 65% by weight, calculated to the total weight of the epoxy resin composition.

The filler material may optionally be coated for example with a silane or a siloxane known for coating filler materials, e.g. dimethylsiloxanes which may be cross linked, or other known coating materials.

The filler material optionally may be present in a “porous” form. As a porous filler material, which optionally may be coated, is understood, that the density of said filler material is within the range of 60% to 80%, compared to the real density of the non-porous filler material. Such porous filler materials have a higher total surface than the non-porous material. Said surface preferably is higher than 20 m²/g (BET m²/g) and preferably higher than 30 m²/g (BET) and preferably is within the range of 30 m²/g (BET) to 100 m²/g (BET), preferably within the range of 30 m²/g (BET) to 60 m²/g (BET).

Preferred curing agents are for example tertiary amines, such as benzyldimethylamine or amine-complexes such as complexes of tertiary amines with boron trichloride or boron trifluoride; urea derivatives, such as N-4-chlorophenyl-N′,N′-dimethylurea (Monuron); optionally substituted imidazoles such as imidazole or 2-phenyl-imidazole. Preferred are tertiary amines, especially 1-substituted imidazole and/or N,N-dimethylbenzylamine, such as 1-alkyl imidazoles which may or may not be substituted also in the 2-position, such as 1-methyl imidazole or 1-isopropyl-2-methyl imidazole. Preferred is 1-methyl imidazole. The amount of catalyst used is a concentration of about 0.05% to 2% by weight, preferably about 0.05% to 1% by weight, calculated to the weight of the DGEBA present within the composition.

Suitable hydrophobic compounds or mixtures of such compounds, especially for improving the self-healing properties of the electrical insulator may be selected from the group comprising flowable fluorinated or chlorinated hydrocarbons which contain —CH₂-units, —CHF-units, —CF₂-units, —CF₃-units, —CHCl-units, —C(Cl)₂-units, —C(Cl)₃-units, or mixtures thereof; or a cyclic, linear or branched flowable organopolysiloxane. Such compounds, also in encapsulated form, are known per se.

The hydrophobic compound preferably has a viscosity in the range from 50 cSt to 10,000 cSt, preferably in the range from 100 cSt to 10,000 cSt, preferably in the range from 500 cSt to 3000 cSt, measured in accordance with DIN 53 019 at 20° C.

Suitable polysiloxanes are known and may be linear, branched, cross-linked or cyclic. Preferably the polysiloxanes are composed of —[Si(R)(R)O]-groups, wherein R independently of each other is an unsubstituted or substituted, preferably fluorinated, alkyl radical having from 1 to 4 carbon atoms, or is phenyl, preferably methyl, and wherein said substituent R may carry reactive groups, such as hydroxyl or epoxy groups. Non-cyclic siloxane compounds preferably on average have about from 20 to 5000, preferably 50-2000, —[Si(R)(R)O]-groups. Preferred cyclic siloxane compounds are those comprising 4-12, and preferably 4-8, —[Si(R)(R)O]-units.

The hydrophobic compound is added to the epoxy resin composition preferably in an amount of from 0.1% to 10%, preferably in an amount of from 0.25% to 5% by weight, preferably in an amount of from 0.25% to 3% by weight, calculated to the weight of the weight of the epoxy resin component present.

The present invention further refers to a method of producing the curable epoxy resin composition as described above, comprising the following steps: (a) preparing the chemically modified polycarbonic acid anhydride hardener component by reacting a polycarbonic acid anhydride as defined above with a glycol or a polyglycol of formula (I) or by reacting a polycarbonic acid anhydride as defined above with a compound containing two carboxylic groups of formula (II), whereby the chemically modified polycarbonic acid anhydride hardener component is obtained, and (b) subsequently mixing said chemically modified polycarbonic acid anhydride hardener component with the epoxy resin component and all the further components and additives which optionally may be present in the epoxy resin composition, optionally under vacuum, in any desired sequence.

The uncured epoxy resin composition is cured at a temperature preferably within the range of 50° C. to 280° C., preferably within the range of 100° C. to 200° C., preferably within the range of 100° C. to 170° C., and preferably at about 130° C. and during a curing time within the range of about 1 hour to about 10 hours. Curing generally is possible also at lower temperatures, whereby at lower temperatures complete curing may last up to several days depending on the catalyst present and its concentration.

Suitable processes for shaping the cured epoxy resin compositions of the invention are for example the APG (Automated Pressure Gelation) Process and the Vacuum Casting Process. Such processes typically include a curing step in the mold for a time sufficient to shape the epoxy resin composition into its final infusible three dimensional structure, typically up to ten hours, and a post-curing step of the demolded article at elevated temperature to develop the ultimate physical and mechanical properties of the cured epoxy resin composition. Such a post-curing step may take, depending on the shape and size of the article, up to thirty hours.

Preferred uses of the insulation systems produced according to the present invention are dry-type transformers, particularly cast coils for dry type distribution transformers, especially vacuum cast dry distribution transformers, which within the resin structure contain metal coils, cores and auxiliary parts of instrument transformers; high-voltage insulations for indoor and outdoor use, like breakers or switchgear applications; high voltage and medium voltage bushings; as long-rod, composite and cap-type insulators, and also for base insulators in the medium-voltage sector, in the production of insulators associated with outdoor power switches, measuring transducers, leadthroughs, and overvoltage protectors, in switchgear constructions, in power switches, and electrical machines, as coating materials for transistors and other semiconductor elements and/or to impregnate electrical components.

The following examples illustrate the invention without limiting the scope of the claimed invention.

### EXAMPLE 1

### Preparation of the Chemically Modified Anhydride Hardener)

85 parts of methyltetrahydrophthalic anhydride (MTHPA, HY 918, from Huntsman Advanced Materials Ltd.) were mixed each time with (a) 5 parts, (b) 7.5 parts, (c) 10 parts, (d) 15 parts, and (e) 20 parts of neopentyl glycol (NPG, 2-dimethyl-1,3-propanediol, CAS No. 126-30-7, from Fluka AG). The mixture was stirred each time in a reaction vessel for two hours at 80° C. In this reaction, NPG is the limiting reagent which results in the formation of a mixture of diester and the unreacted anhydride. The composition of the obtained mixture is given in Table 1.

TABLE 1

| | | | HEW* of |
|---|---|---|---|
| | | unreacted | modified |
| 85 parts MTHPA | diester | anhydride | anhydride |
| reacted with: | (% by weight) | (% by weight) | (g/equiv.) |
| (a) 5 parts NPG | 23.3 | 76.7 | 175.9 |
| (b) 7.5 parts NPG | 34.0 | 66.0 | 180.8 |
| (c) 10 parts NPG | 44.1 | 55.9 | 185.7 |
| (d) 15 parts NPG | 62.9 | 37.1 | 195.5 |
| (e) 20 parts NPG | 79.8 | 20.2 | 205.27 |

*HEW = Hardener Equivalent Weight

### EXAMPLE 2

Stoichiometric amounts of the modified hardener as prepared in Example 1(d) [85 parts MTHPA reacted with 15 parts of NPG] based on its Hardener Equivalent Weight (HEW) was used for curing the conventional DGEBA epoxy resin composition. A comparative Example was carried out using the generic non-modified anhydride based hardener. The compositions of the DGEBA epoxy resin compositions are given in Table 2 below.

TABLE 2
(Epoxy formulation with 15 phr NPG modified anhydride
and Comparative Example)

| | Hardener from | Comparative |
|---|---|---|
| | Example 1 | Example |
| Components | (phr) | (phr) |
| Araldite ® CY 228-1 (Resin) | 100 | 100 |
| Modified hardener | 100 | -.- |
| (85 phr HY 918 reacted | | |
| with 15 phr NPG) | | |
| Conventional hardener | -.- | 85 |
| HY 918 | | |
| DY 045 (Flexibiliser) | 20 | 20 |
| DY 062 (Catalyst) | 0.5 | 0.5 |
| TD 202 (Colour) | 1.5 | 1.5 |
| W12 (Filler) | 412 | 412 |

phr: components expressed in parts per hundred of the resin (phr)
CY 228-1: DGEBA based liquid epoxy resin, Huntsman Advanced Materials Ltd.
HY 918: MTHPA (Huntsman Advanced Materials Ltd.)
DY 045: Polyethlene glycol, n = about 15, (Huntsman Advanced Materials Ltd.)
DY 062: Benzyldimethylamine (BDMA), (Huntsman Advanced Materials Ltd.)
W12: Silica filler, particle size of the order 10⁻² mm

Tested formulations cured with the modified hardener as per Example 1 were tested for low temperature cracking resistance with temperature ranging from −10° C. to −70° C. The modified hardener from Example 1 enabled the cured epoxy to pass low temperature cracking test until minus 70° C. This is in contrast with the generic 65 wt. % silica filled, DGEBA-anhydride cured epoxy (Comparative Example) which fails at −40° C. Moreover, the cured composition according to Example 1 retained the mechanical, thermal and electrical properties.

### EXAMPLE 3

Analogous results are obtained in Example 2 when replacing the modified hardener from Example 1(c) by the modified Hardeners from Example 1(a), Example 1(b), Example 1(c) and Example 1(e).

### EXAMPLE 4

Analogous results are obtained in Example 2 when replacing the epoxy resin component (CY 228-1) by another epoxy resin component such as DGEBA epoxy resin component with a different epoxy value than CY 228-1 or by DGEBF, or by the cycloaliphatic epoxy resin component Araldite® 184, or Araldite® CY 5622, both from Huntsman Advanced Materials Ltd.

1. Curable epoxy resin composition comprising at least an epoxy resin component and a hardener component, and optionally further additives, wherein:
(a) at least a part of the hardener component is a chemically modified polycarbonic acid anhydride, said chemically modified polycarbonic acid anhydride being a reaction product of a polycarbonic acid anhydride and a glycol (diol) or a polyglycol, or a reaction product of a polycarbonic acid anhydride and a compound containing two carboxylic groups;
(b) said glycol or polyglycol being selected from a group consisting of compounds which contain formula (I):
HO—(CₙH₂ₙ—O)ₘ—H (I)
wherein n is an integer from 2 to 8; and m is an integer from 1 to 10;
(c) said compound containing two carboxylic groups being selected from a group consisting of compounds which contain formula (II):
HOOC—(CₚH₂ₚ)—COOH (II)
wherein p an integer from 2 to 18; and
(d) the chemically modified acid anhydride hardener is present in an amount of at least 10% of the reactive hardening groups calculated to all the reactive hardening groups contained in a total amount of hardener component present in the epoxy resin composition.

2. Curable epoxy resin composition according to claim 1, wherein the epoxy resin component contains at least two 1,2-epoxy groups per molecule and is a cycloaliphatic and/or aromatic epoxy resin compound comprising unsubstituted glycidyl groups and/or glycidyl groups substituted with methyl groups, wherein said epoxy resin component has an epoxy value (equiv./kg) of at least three.

3. Curable epoxy resin composition according to claim 2, wherein the epoxy resin component is an optionally substituted compound of formula (III): 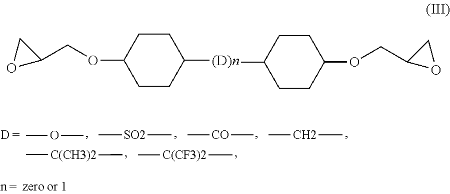
or optionally substituted compound of formula (IV): 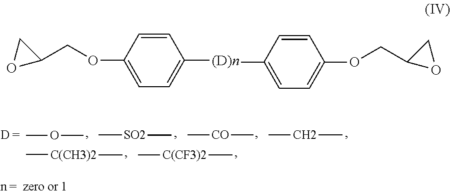
and wherein D is: —(CH₂)— or [—C(CH₃)₂—].

4. Curable epoxy resin composition according to claim 3, wherein the epoxy resin component is hexahydro-o-phthalic acid-bis-glycidyl ester, hexahydro-m-phthalic acid-bis-glycidyl ester and/or hexahydro-p-phthalic acid-bis-glycidyl ester.

5. Curable epoxy resin composition according to claim 1, wherein the total amount of hardener component is composed of a chemically non-modified hardener compound and the chemically modified hardener compound.

6. Curable epoxy resin composition according to claim 5, wherein the chemically non-modified hardener compound is an aliphatic and/or cycloaliphatic polycarbonic acid anhydride.

7. Curable epoxy resin composition according to claim 1, wherein the chemically modified polycarbonic acid anhydride has been made prior to adding to the epoxy resin composition and has been made from an aliphatic and/or cycloaliphatic polycarbonic acid anhydride by reaction with a compound of formula (I) or formula (II).

8. Curable epoxy resin composition according to claim 1, wherein in formula (I) n is 2, 3, 4, 5 or 6, and m is an integer from 1 to 10.

9. Curable epoxy resin composition according to claim 1, wherein in formula (II) p is an integer from 2 to 16.

10. Curable epoxy resin composition according to claim 1, wherein the chemically modified acid anhydride hardener has been made by reacting an aliphatic and/or cycloaliphatic polycarbonic acid anhydride.

11. Curable epoxy resin composition according to claim 1, wherein the chemically modified acid anhydride hardener is present in an amount comprising 10% to 100% of the reactive hardening groups, calculated to all the reactive hardening groups contained in the total amount of hardener component present in the epoxy resin composition.

12. Curable epoxy resin composition according to claim 1, wherein the total amount of hardener component containing the reactive hardening groups is present within the epoxy resin composition in a concentration within a range of 0.2 to 1.2 equivalents of reactive hardening groups per epoxy equivalent present in the epoxy resin composition.

13. Curable epoxy resin composition according to claim 1, wherein said curable epoxy resin composition comprises:
a filler material, a curing agent for enhancing the polymerization of the epoxy resin with the hardener and optionally one or more further additives selected from hydrophobic compounds, wetting/dispersing agents, plasticizers, antioxidants, light absorbers, pigments, flame retardants, fibers and other additives generally used in electrical applications.

14. Curable epoxy resin composition according to claim 13, wherein said inorganic filler is present within the range of about 50% by weight to about 80% by weight calculated to the total weight of the epoxy resin composition.

15. Curable epoxy resin composition according to claim 14, wherein the density of said filler material is within the range of 60% to 80%, compared to the real density of the non-porous filler material.

16. The chemically modified polycarbonic acid anhydride hardener component according to claim 7.

17. Method of making the hardener component according to claim 7, comprising:
reacting at least 50% of the chemically non-modified hardener compound, said chemically non-modified hardener compound being an aliphatic and/or cycloaliphatic polycarbonic acid anhydride, with a compound of formula (I), or with a compound of formula (II), wherein the hydroxyl-equivalents of the compound of the formula (I) or the carboxyl-equivalents of the compound of the formula (II) present within the reaction mixture are within the range of 10% to 100%, calculated to the total amount of reactive hardening groups present within the total amount of hardener component.

18. Method of making the curable epoxy resin composition according to claim 1, comprising:
(a) preparing the chemically modified polycarbonic acid anhydride hardener component by reacting a polycarbonic acid anhydride, with a glycol or a polyglycol of formula (I), by reacting a polycarbonic acid anhydride with a compound containing two carboxylic groups of formula (II), and
(b) subsequently mixing the obtained chemically modified polycarbonic acid anhydride hardener component with the epoxy resin component and all the further components and additives which optionally may be present in the epoxy resin composition in any desired sequence.

19. The curable epoxy resin composition according to claim 1 in combination with an insulation system in an electrical article.

20. The composition according to claim in further combination with at least one of dry-type transformers, especially vacuum cast dry distribution transformers, which within the resin structure contain metal coils, cores and auxiliary parts of instrument transformers; high-voltage insulations for indoor and outdoor use; high voltage and medium voltage bushings; as long-rod, composite and cap-type insulators, and also for base insulators in a medium-voltage sector, in production of insulators associated with outdoor power switches, measuring transducers, leadthroughs, and overvoltage protectors, in switchgear constructions, in power switches, and electrical machines, as coating materials for transistors and other semiconductor elements and/or to impregnate electrical components.

21. The cured epoxy resin composition obtained from a curable epoxy resin composition according to claim 1, wherein said cured epoxy resin composition is present in the form of an electrical insulation system.

22. An electrical article comprising an electrical insulation system made from a curable epoxy resin composition according to claim 1.
